SEQUENCE LISTING (1 sequence)

SEQ ID NO: 1 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

(19) United States
(12) Patent Application Publication
(10) Pub. No.: US 2021/0261936 A1
(43) Pub. Date: Aug. 26, 2021

(54) COMPOSITIONS AND METHODS FOR ENHANCING THE UBIQUITIN PROTEASOME SYSTEM

(71) Applicant: South Dakota Board of Regents, Pierre, SD (US)

(72) Inventor: Xuejun Wang, Vermillion, SD (US)

(21) Appl. No.: 17/184,187

(22) Filed: Feb. 24, 2021

Related U.S. Application Data
(60) Provisional application No. 62/980,654, filed on Feb. 24, 2020.

(51) Int. Cl. C12N 9/50; A61K 47/68

(52) U.S. Cl. CPC C12N 9/50; A61K 47/6815; A61K 38/00; C07K 2319/33; C07K 2319/10; C12Y 304/24011

(57) ABSTRACT

Disclosed herein is a method of enhancing the ubiquitin proteasome system (“UPS”) in a subject in need thereof, comprising administering an effective amount of a composition comprising neprilysin and a cell targeting moiety, wherein administration of the composition delivers the composition into the intracellular space of one or more cells of the subject and wherein the subject suffers from a condition associated with a UPS deficiency.

### CROSS-REFERENCE TO RELATED APPLICATION(S)

This application claims priority to U.S. Provisional Application No. 62/980,654 filed Feb. 24, 2020 and entitled “COMPOSITIONS AND METHODS FOR ENHANCING THE UBIQUITIN PROTEASOME SYSTEM,” which is hereby incorporated by reference in its entirety under 35 U.S.C. § 119(e).

### TECHNICAL FIELD

The disclosure relates to methods for enhancing the performance of the Ubiquitin Proteasome System.

### BACKGROUND

The Ubiquitin Proteasome System (“UPS”) is responsible for the degradation of more than 80% of cellular proteins, normal or abnormal. UPS functional insufficiency is linked to the genesis and progression of a large subset of human diseases, such as neurodegenerative disease, a large subset of heart failure, and diabetes. Prior preclinical studies have demonstrated compellingly that genetic enhancement of UPS function can be therapeutically beneficial to the treatment of these diseases.

Accordingly, there is a need in the art for compositions and methods to enhance UPS in subjects with diseases characterized by UPS deficiencies.

### BRIEF SUMMARY

Disclosed herein is a method of enhancing the ubiquitin proteasome system (“UPS”) in a subject in need thereof, comprising administering an effective amount of a composition comprising neprilysin and a cell targeting moiety, wherein administration of the composition delivers the composition into the intracellular space of one or more cells of the subject and wherein the subject suffers from a condition associated with a UPS deficiency. In certain aspects, the cell targeting moiety facilitates entry of the composition into the one or more cells of the subject. In certain implementations, the cell targeting moiety comprises an antibody fragment. In further aspects, the neprilysin and antibody fragment comprise a fusion protein. According to certain alternative implementations, the cell targeting moiety comprises a HIV-1 TAT-derived cell-penetrating peptide.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A depicts representative images of a western blot analysis for GFPu and RFP in NRCMs, according to certain embodiments.

FIG. 1B shows pooled densitometry data relating to the NRCM cultures of FIG. 1A.

FIG. 2A depicts representative images of a western blot analysis for GFPu and RFP levels in NRCMs with Fab-NEP treatment followed by cycloheximide (CHX) treatment, according to certain embodiments.

FIG. 2B shows the quantified and normalized data from FIG. 2A.

FIG. 3A shows Western blot data indicating enhanced UPS proteolytic function in mice, according to certain embodiments.

FIG. 3B shows quantification of the data shown in FIG. 3A.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more cancer disorders prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, administration to specific organs through invasion, intramuscular administration, intratumoral administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Effective dosages may be estimated initially from in vitro assays. For example, an initial dosage for use in animals may be formulated to achieve a circulating blood or serum concentration of active compound that is at or above an IC50 of the particular compound as measured in an in vitro assay. Calculating dosages to achieve such circulating blood or serum concentrations, taking into account the bioavailability of the particular active agent, is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, which is hereby incorporated by reference in its entirety, and the references cited therein.

Disclosed herein is the discovery that a cell penetrating form of Neprilysin (NEP) can enhance UPS-mediated protein degradation, developing a new method to enhance UPS performance that can be used for treating forms of diseases where UPS function inadequacy plays a pathogenic role.

Without wishing to be bound to any particular theory, the inventors have discovered that when delivered into the cytoplasm of the cell, NEP is capable of enhancing the proteolytic function of the UPS. This provides a new method to enhance UPS performance to treat common forms of diseases in which UPS function inadequacy plays a role. NEP is a primarily a cell membrane bound Zn-dependent endopeptidase although in soluble form is also present in extracellular space; hence the endogenous natural forms of NEP by design act to cleave small peptides in the extracellular space.

Disclosed herein is a method of enhancing the UPS in a subject in need thereof, comprising administering an effective amount of a composition comprising NEP and a cell targeting moiety, wherein administration of the composition delivers the composition into the intracellular space of one or more cells of the subject. In certain aspects, the cell targeting moiety facilitates entry of the composition into the one or more cells of the subject. In certain embodiments, the cell targeting moiety facilitates entry of the composition into a specific cell type of the body of the subject (e.g., cardiomyocytes).

In certain implementations, the cell targeting moiety comprises an antibody fragment. In certain embodiments, the antibody fragment is from a humanized antibody. In exemplary implementations, the antibody fragment is a Fab fragment. A “Fab fragment” is comprised of one light chain and the CH1 and variable regions of one heavy chain. Generally, the heavy chain of a Fab molecule cannot form a disulfide bond with another heavy chain molecule. A Fab may optionally include a portion of the hinge, such as the upper hinge.

In certain embodiments, the cell targeting moiety comprises a humanized Fab fragment from an anti-dsDNA antibody, 3E10. A description of the composition and use of such Fab fragments is described in U.S. Pat. No. 10,221,250, which hereby incorporated by reference in its entirety.

According to certain alternative implementations, the cell targeting moiety comprises a HIV-1 TAT-derived cell-penetrating peptide.

In further aspects, the NEP and cell targeting moiety comprise a fusion protein. In some embodiments, conjugation between the NEP and cell targeting moiety is accomplished by generating a fusion protein containing a NEP and cell targeting moiety, expressed as one contiguous polypeptide chain. In preparing such fusion proteins, a fusion gene is constructed comprising nucleic acids which encode a NEP polypeptide and cell targeting moiety, and optionally, a peptide linker sequence to span the NEP polypeptide and the cell targeting moiety. The disclosure contemplates that suitable complexes, such as fusion proteins, may be in either orientation. In other words, the cell targeting moiety portion may be N-terminal or C-terminal to the NEP polypeptide.

In certain embodiments, the NEP polypeptide component of the NEP-cell targeting moiety fusion protein comprises a polypeptide with an amino acid sequence of SEQ ID NO 1. In further embodiments, comprising an amino acid sequence that is at least 90% (or at least 95%, 96%, 97%, 98%, 99% or 100%) identical to the amino acid sequence set forth in SEQ ID NO 1.

In certain implementations, the composition further comprises a cell-type specific tag. As used herein, cell-type specific tag means a moiety attached to the composition that confers the ability to preferentially bind a specific cell type relative to other cell-types of the subject.

According to certain aspects, the subject suffers from a disease characterized by a UPS deficiency. In exemplary implementations, the administration of the composition increases degradation of misfolded proteins within the one or more cells of the subject. In certain implementations, misfolded proteins are degraded prior to aggregation. In further aspects, administration of the composition inhibits the aggregation of misfolded proteins. In exemplary implementations of these embodiments, the administration of the composition inhibits the formation of pre-amyloid oligomers.

As mentioned, in certain aspects, the subject suffers from a disease characterized by a UPS deficiency. Such conditions are characterized by soluble or insoluble aggregates of misfolded or otherwise aberrant proteins within the cell. In exemplary implementations, the subject suffers from a neurodegenerative disease. Such diseases include, but are not limited to Alzheimer's disease, Parkinson's disease, amyotrophic lateral sclerosis (ALS), Huntington's disease, transmissible spongiform encephalopathies (TSEs), Creutzfeld-Jakob disease, systemic amyloidosis, prion-based diseases, and diseases caused by polyglutamine repeats.

In exemplary implementations, administration of the composition inhibits the formation of pre-amyloid oligomers.

According to certain embodiments, the subject suffers from heart disease. In exemplary implementations, the heart disease is heart failure with preserved ejection fraction (HFpEF). In alternative implementations, the heart disease is heart failure with reduced ejection fraction (HFrEF). In further implementations, the heart disease is myofibrillar myopathy. In yet further implementations, the heart disease is characterized by cardiac proteotoxicity.

According to certain embodiments, the composition is administered to the subject in a prophylactically effective amount. In exemplary implementations of these embodiments, the subject is at high risk of developing a disease associated with aggregates of misfolded proteins (e.g., a UPS deficiency disorder).

In certain aspects, the administration of the composition is periodic. In further aspects, the method further includes the step of intermittently reassessing the disease state of the subject and readjusting the periodic dose based on the assessment of the disease state.

In certain aspects, the subject has been diagnosed with diabetes.

Further disclosed herein is a method of enhancing the UPS in a subject in need thereof, comprising administering an effective amount of viral vector and a heterologous nucleic acid encoding the polypeptide neprilysin. In certain aspects, the viral vector is delivered to one or more cells containing misfolded protein aggregates or depositions.

Depending upon the subject to be treated and the route of administration, the compounds of the invention may be administered at varying doses. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses. More preferred daily doses are in the range 2.5 to 250 mg (e.g., about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, or about 250 mg and the like or any range or value therein) per subject.

Individual doses of compounds of the invention may be in the range 1 to 100 mg (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, and the like, or any range or values therein). Advantageously, compounds of the present invention may be administered in single doses, e.g. once daily or more seldom, or in a total daily dosage administered in divided doses of two, three or four times daily.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

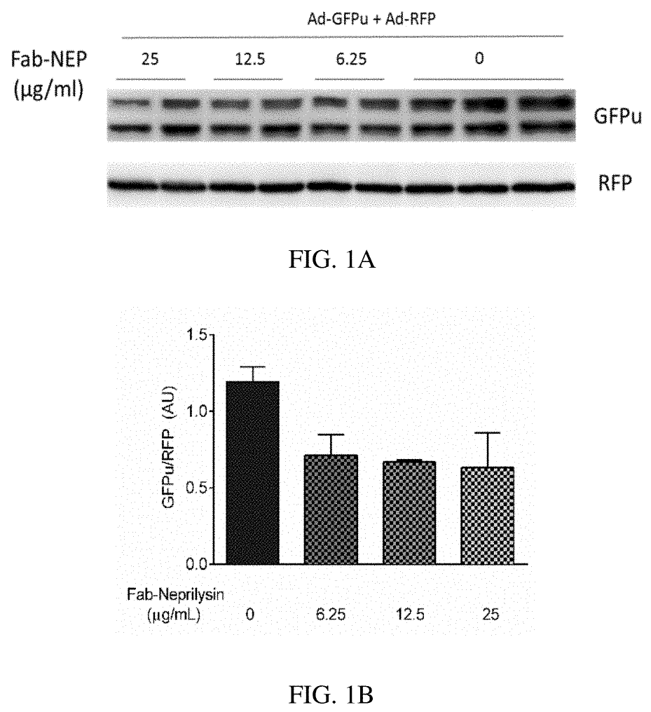

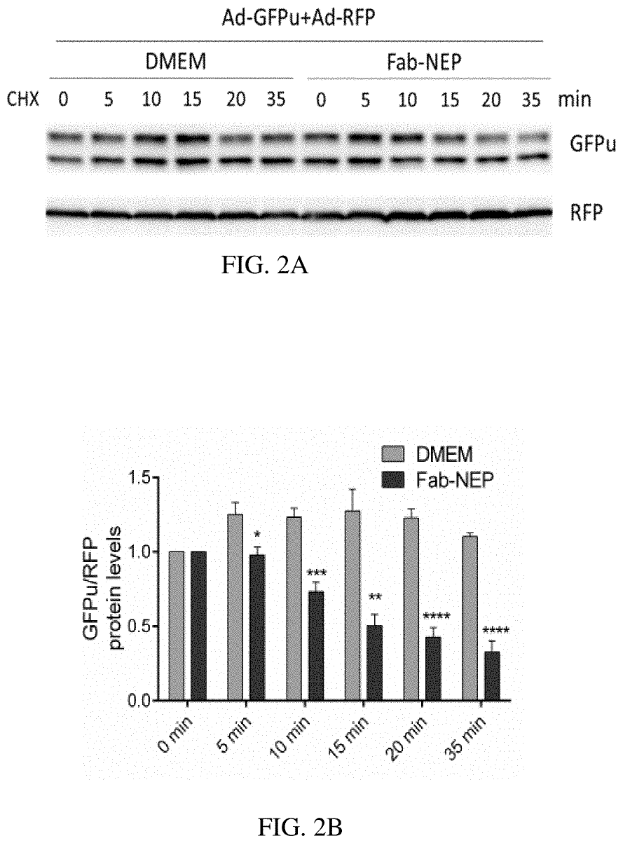

A fusion protein of NEP and an antibody fragment was created (referred to herein as “Fab-NEP”) to test effects of intracellular delivery of NEP on UPS function. FIG. 1 shows Fab-NEP reduced GFPu/RFP ratio in cultured cardiomyocytes, which indicates UPS performance can be likely enhanced by Fab-NEP. FIG. 2 shows data that indicates the half-life of GFPu in cultured cardiomyocytes were shortened by Fab-NEP treatment, further confirming that Fab-NEP enhances UPS performance.

A CHX-chase test showed indeed the half-life of GFPu (an inverse reporter of UPS function) was shortened by Fab-NEP, which confirms that Fab-NEP promotes UPS function via degradation of a surrogate misfolded protein.

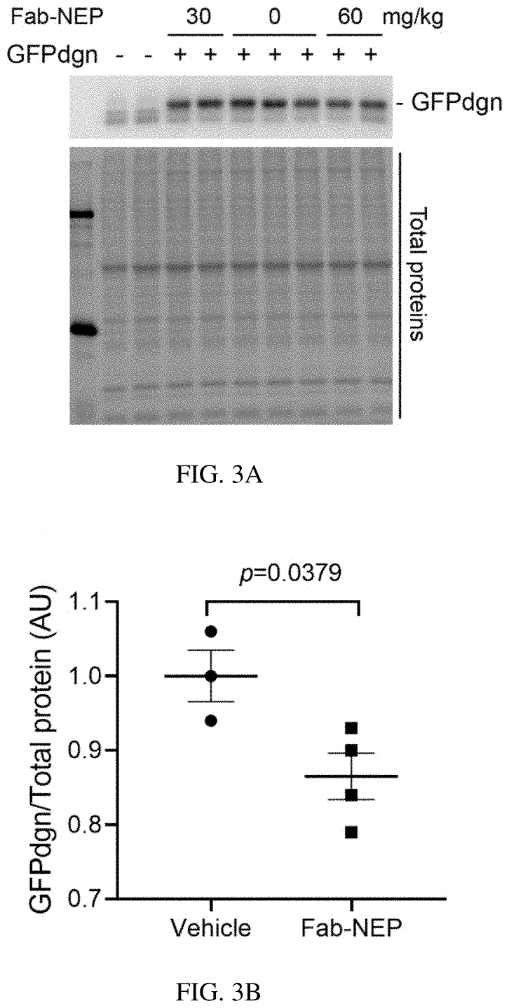

To confirm whether the UPS enhancement effect of Fab-NEP observed in cell cultures also occurs in intact animals, studies were conducted on adult transgenic mice that ubiquitously and constitutively express GFPdgn, a transgenic protein that is similar to the GFPu used in the cell culture experiments and is a proven inverse reporter of UPS performance inside the cell and a surrogate misfolded protein expressed inside the cell. GFPdgn transgenic mice were subjected to an intraperitoneal injection of either Fab-NEP (30 or 60 mg/kg body weight) or the equivalent volume of vehicle control (20 mM HEPES, 140 mM NaCl, at pH 7.5). At 6 hours after the injection, the animals were sacrificed and the heart tissue samples were collected for total protein extraction and Western blot analysis for the protein level of the transgenic GFPdgn (FIG. 3). The total protein image obtained with the stain-free total protein imaging technology from the gel used for the Western blot analysis was used as the in-lane loading control for the measurement of GFPdgn protein levels. Mice treated with Fab-NEP at the dose of 30 mg/kg (2 mice) and of 60 mg/kg (2 mice) were combined into the Fab-NEP treated group for the statistical analysis. The p value is derived from unpaired Welch's t-test.

As shown in FIG. 3, these data indicate that the Fab-NEP treatment leads to a significant decrease in myocardial GFPdgn proteins, demonstrating that Fab-NEP administered via intraperitoneal injection enhances the proteolytic function of the UPS in intact animals.

### FIGURE CAPTIONS

FIGS. 1A and B. Fab-NEP treatment decreases the protein levels of an inverse reporter for the UPS in cultured neonatal rat cardiomyocytes (NRCMs). NRCMs in cultures were infected with a mixture of recombinant adenoviruses harboring the expression cassette of GFPu (Ad-GFPu) or RFP (Ad-RFP) and then treated with vehicle (DMEM) or Fab-NEP at the indicated doses for 48 hours before harvested for protein extraction and western blot analyses for GFPu and RFP. Shown are representative images (FIG. 1A) and pooled densitometry data (FIG. 1B).

FIGS. 2A and B. Cycloheximide (CHX) chase assays reveal enhanced UPS-mediated degradation of GFPu, a known UPS substrate, by Fab-NEP treatment in cultured NRCMs. NRCMs in cultures were infected with a mixture of Ad-GFPu and Ad-RFP for 28 hours before treated with vehicle (DMEM) or 10 μg/mL Fab-NEP for 24 hours. Cells were then dosed with CHX to prevent further protein synthesis. Samples were taken after the indicated time in minutes (min). Representative images (FIG. 2A) and pooled densitometry data (FIG. 2B) of western blot analyses for GFPu and RFP levels are shown. Mean±SEM, n=3 biological repeats; *p<0.05, **p<0.01, ***p<0.005; multiple t-tests with the Holm-Sidak correction.

FIGS. 3A and B. Fab-NEP enhances UPS proteolytic function in mice. Adult GFPdgn transgenic mice (GFPdgn+) were subjected to an intraperitoneal injection of either Fab-NEP (30 or 60 mg/kg body weight) or the equivalent volume of vehicle control (n=3 mice). At 6 hours after the injection, the animals were sacrificed and the ventricular myocardial samples were collected and processed for total protein extraction and Western blot analysis for the protein level of the transgenic GFPdgn. FIG. 3A shows a representative image of the Western blot analysis for GFPdgn (upper image) and the stain-free gel image of total proteins (bottom image) that was used as the in-lane loading control. FIG. 3B shows a scatter dot plot superimposed by mean±SEM of the densitometry data derived from the images shown in panel A. Each lane (in FIG. 3A) or each dot (in FIG. 3B) represents a unique mouse. Mice treated with Fab-NEP at either dose (30 or 60 mg/kg) are combined into the Fab-NEP group (n=4 mice) for the statistical analysis (B). The p value is derived from unpaired Welch's t-test.

Although the present disclosure has been described with references to various embodiments, persons skilled in the art will recognized that changes may be made in form and detail without departing from the spirit and scope of this disclosure.

Sequence Information
SEQ ID NO: 1-Neprilysin Isoform a
NP_001341572.1
MGKSESQMDITDINTPKPKKKQRWTPLEISLSVLVLLLTIIAVTMIALYA
TYDDGICKSSDCIKSAARLIQNMDATTEPCTDFFKYACGGWLKRNVIPET
SSRYGNFDILRDELEVVLKDVLQEPKTEDIVAVQKAKALYRSCINESAID
SRGGEPLLKLLPDIYGWPVATENWEQKYGASWTAEKAIAQLNSKYGKKVL
INLFVGTDDKNSVNHVIHIDQPRLGLPSRDYYECTGIYKEACTAYVDFMI
SVARLIRQEERLPIDENQLALEMNKVMELEKEIANATAKPEDRNDPMLLY
NKMTLAQIQNNFSLEINGKPFSWLNFTNEIMSTVNISITNEEDVVVYAPE
YLTKLKPILTKYSARDLQNLMSWRFIMDLVSSLSRTYKESRNAFRKALYG
TTSETATWRRCANYVNGNMENAVGRLYVEAAFAGESKHVVEDLIAQIREV
FIQTLDDLTWMDAETKKRAEEKALAIKERIGYPDDIVSNDNKLNNEYLEL
NYKEDEYFENIIQNLKFSQSKQLKKLREKVDKDEWISGAAVVNAFYSSGR
NQIVFPAGILQPPFFSAQQSNSLNYGGIGMVIGHEITHGFDDNGRNFNKD
GDLVDWWTQQSASNFKEQSQCMVYQYGNFSWDLAGGQHLNGINTLGENIA
DNGGLGQAYRAYQNYIKKNGEEKLLPGLDLNHKQLFFLNFAQVWCGTYRP
EYAVNSIKTDVHSPGNFRIIGTLQNSAEFSEAFHCRKNSYMNPEKKCRVW

What is claimed is:

1. A method of enhancing the ubiquitin proteasome system (“UPS”) in a subject in need thereof, comprising administering an effective amount of a composition comprising neprilysin and a cell targeting moiety, wherein administration of the composition delivers the composition into the intracellular space of one or more cells of the subject.

2. The method of claim 1, wherein the cell targeting moiety facilitates entry of the composition into the one or more cells.

3. The method of claim 2, wherein the cell targeting moiety comprises an antibody fragment.

4. The method of claim 3, wherein neprilysin and antibody fragment comprise a fusion protein.

5. The method of claim 2, wherein the cell targeting moiety comprises a HIV-1 TAT-derived cell-penetrating peptide.

6. The method of claim 1, wherein the composition further comprises a cell-type specific tag.

7. The method of claim 1, wherein administration of the composition increases degradation of misfolded proteins within the one or more cells.

8. The method of claim 7, wherein misfolded proteins are degraded prior to aggregation.

9. The method of claim 7, wherein administration of the composition inhibits the aggregation of misfolded proteins.

10. The method of claim 7, wherein the administration of the composition inhibits the formation of pre-amyloid oligomers.

11. The method of claim 1, wherein the subject suffers from a disease characterized by a UPS deficiency.

12. The method of claim 11, wherein the subject suffers from a neurodegenerative disease.

13. The method of claim 11, wherein the administration of the composition inhibits the formation of pre-amyloid oligomers.

14. The method of claim 11, wherein the subject suffers from heart disease.

15. The method of claim 14, wherein the heart disease is heart failure with preserved ejection fraction (HFpEF).

16. The method of claim 14, wherein the heart disease is heart failure with reduced ejection fraction (HFrEF).

17. The method of claim 14, wherein the heart disease is myofibrillar myopathy.

18. The method of claim 14, wherein the heart disease is characterized by cardiac proteotoxicity.

19. The method of claim 1, wherein the subject is at high risk of developing a disease associated with aggregates of misfolded proteins.

20. A method of enhancing the UPS in a subject in need thereof, comprising administering an effective amount of viral vector and a heterologous nucleic acid encoding the polypeptide neprilysin.
